(19) United States
(12) Patent Application Publication
(10) Pub. No.: US 2022/0031245 A1
(43) Pub. Date: Feb. 3, 2022

(54) SLEEVE WITH CONFIGURABLE ELECTRODES FOR FUNCTIONAL ELECTRICAL STIMULATION AND/OR ELECTROMYOGRAPHY

(71) Applicant: Battelle Memorial Institute, Columbus, OH (US)

(72) Inventor: Herbert S. Bresler, Bexley, OH (US)

(21) Appl. No.: 17/389,437

(22) Filed: Jul. 30, 2021

Related U.S. Application Data
(60) Provisional application No. 63/058,792, filed on Jul. 30, 2020.

(51) Int. Cl. A61B 5/00; A61B 5/256; A61B 5/389; A61N 1/04

(52) U.S. Cl. CPC A61B 5/6824; A61B 5/256; A61B 5/389; A61B 2562/12; A61N 1/0452; A61N 1/0456; A61B 2562/043; A61N 1/0484

(57) ABSTRACT

A device for functional electrical stimulation (FES), neuromuscular electrical stimulation (NMES), and/or electromyography (EMG) readout includes a sleeve sized and shaped to be worn on a human arm and comprising an inner sleeve and an outer sleeve. The inner sleeve has openings formed therein, and has an exposed side positioned to contact skin and an opposite backside facing the outer sleeve. Electrode strips each comprise a linear circuit board on which a row of electrodes is mounted. The electrode strips are detachably and selectively mountable to the inner sleeve in a plurality of different orientations. The electrode strips are mountable on the inner sleeve with the linear circuit boards disposed on the backside of the inner sleeve between the inner sleeve and the outer sleeve and the electrodes passing through the openings of the inner sleeve so as to be positioned to contact skin.

This application claims the benefit of U.S. Provisional Application No. 63/058,792 filed Jul. 30, 2020 and titled “SLEEVE WITH CONFIGURABLE ELECTRODES FOR FUNCTIONAL ELECTRICAL STIMULATION AND/OR ELECTROMYOGRAPHY”. U.S. Provisional Application No. 63/058,792 filed Jul. 30, 2020 and titled “SLEEVE WITH CONFIGURABLE ELECTRODES FOR FUNCTIONAL ELECTRICAL STIMULATION AND/OR ELECTROMYOGRAPHY” is incorporated herein by reference in its entirety.

### BACKGROUND

The following relates to the neuromuscular electrical stimulation (NMES) arts, functional electrical stimulation (FES) arts, electromyography (EMG) measurement arts, and to related applications such as rehabilitative or assistive systems, to virtual reality (VR) gaming user interfaces, augmented reality (AR) assistive system user interfaces, VR or AR systems employing such user interfaces, and to related arts.

EMG measurement entails measuring electromyography signals generated by musculature. EMG measurement devices are thus devices for receiving user input. That input may be volitional input, where the subject intentionally generates the EMG signals; or may be non-volitional input, for example a case in which a subject suffering from Parkinson's disease may involuntarily generate EMG signals due to pathological tremors. EMG signals may also include a combination of volitional and non-volitional signals, e.g. the aforementioned Parkinson's patient may generate volitional EMG due to intentional movement of an arm that is accompanied by non-volitional EMG due to tremors.

FES and NMES are techniques for applying electrical signals to musculature to generate somatosensory perceptions such as the sensation of being touched, sensation of heat, pain, pressure, or so forth; and/or to stimulate contraction of muscles. In VR or AR systems for gaming or other applications, such generation of somatosensory perceptions can enhance the immersive experience. For patients with muscle debilitation or paralysis due to stroke, spinal cord injury, or other pathology, stimulation of muscle contraction can provide a way to artificially recover muscle activity.

In such systems, the EMG signal readout or FES or NMES application is by way of surface electrodes contacting the skin, or by way of transcutaneous electrodes that penetrate the skin. Surface electrodes are advantageously non-invasive and are preferable or even mandatory in applications such as VR gaming where the user is unlikely to be willing to have electrodes implanted in order to play the game. A wearable sleeve with surface electrodes on the inside surface contacting the skin is a convenient and efficient way to quickly place a large number of electrodes onto the skin.

U.S. Pub. No. 2018/0154133 A1 published Jun. 7, 2018 and filed Jan. 16, 2018, titled “Neural Sleeve for Neuromuscular Stimulation, Sensing and Recording” is incorporated herein by reference in its entirety, and provides some nonlimiting illustrative examples of wearable sleeves with electrodes for NMES, FES, and/or EMG. Disclosed herein are certain improvements.

### BRIEF SUMMARY

In accordance with some illustrative embodiments disclosed herein, a device is disclosed for use in performing FES, in performing NMES, and/or in receiving EMG signals. The device comprises a sleeve and electrode strips. The sleeve is sized and shaped to be worn on a human arm and comprises an inner sleeve and an outer sleeve. The inner sleeve has openings formed therein and has an exposed side positioned to contact skin of the human arm when the sleeve is worn on the human arm and an opposite backside facing the outer sleeve. The electrode strips each comprise a linear circuit board on which a row of electrodes is mounted. The electrode strips are mounted on the inner sleeve with the linear circuit boards disposed on the backside of the inner sleeve between the inner sleeve and the outer sleeve and the electrodes passing through the openings of the inner sleeve so as to be positioned to contact skin of the human arm when the sleeve is worn on the human arm.

In accordance with some illustrative embodiments disclosed herein, a method of assembling a device is disclosed. The device comprises a sleeve having an inner sleeve and an outer sleeve, in which the inner sleeve has an exposed side positioned to contact skin of the human arm when the sleeve is worn on the human arm and an opposite backside facing the outer sleeve. The device is for use in performing FES, NMES, and/or receiving EMG signals. The method comprises securing electrode strips each comprising a linear circuit board on which a row of electrodes is mounted to the inner sleeve. The electrode strips are secured to the inner sleeve with the linear circuit boards disposed on the backside of the inner sleeve between the inner sleeve and the outer sleeve and the electrodes passing through the openings of the inner sleeve so as to be positioned to contact skin of the human arm when the sleeve is worn on the human arm.

In accordance with some illustrative embodiments disclosed herein, a device is disclosed for use in performing FES, in performing NMES, and/or in receiving EMG signals. The device comprises a sleeve and electrode strips. The sleeve is sized and shaped to be worn on a human arm and comprises an inner sleeve and an outer sleeve. The inner sleeve has openings formed therein, and has an exposed side positioned to contact skin of the human arm when the sleeve is worn on the human arm and an opposite backside facing the outer sleeve. The electrode strips each comprise a linear circuit board on which a row of electrodes is mounted. The electrode strips are detachably and selectively mountable to the inner sleeve in a plurality of different orientations. The electrode strips are mountable on the inner sleeve with the linear circuit boards disposed on the backside of the inner sleeve between the inner sleeve and the outer sleeve and the electrodes passing through the openings of the inner sleeve so as to be positioned to contact skin of the human arm when the sleeve is worn on the human arm.

### BRIEF DESCRIPTION OF THE DRAWINGS

FIG. 1 diagrammatically illustrates a perspective view of a device for providing NMES or FES stimulation and/or EMG readout, in combination with driving/control hardware.

FIG. 2 illustrates the inside surface of the opened sleeve, with the electrodes installed.

FIG. 3 illustrates the backside of the inner sleeve with electrode strips installed.

FIG. 4 diagrammatically illustrates an exploded perspective view of the inner and outer sleeves.

FIG. 5 illustrates a side-sectional view depicting the attachment of the electrodes to the inner sleeve.

FIG. 6 diagrammatically illustrates a configurable electrodes arrangement employing electrode strips oriented in different directions. The right-hand side depicts the electrode strips in isolation, while the left-hand side depicts the backside of the inner sleeve with the electrode strips mounted on the inner sleeve.

FIG. 7 diagrammatically illustrates a configurable electrodes arrangement in which a first set of electrode strips are installed in parallel.

FIG. 8 diagrammatically illustrates a configurable electrodes arrangement including the first set of electrode strips of FIG. 7 and further including a second set of electrode strips oriented orthogonally to the first set of electrode strips.

FIG. 9 diagrammatically illustrates a configurable electrodes arrangement employing electrode strips aligned with and mounted to rows of openings in the inner sleeve. The right-hand side depicts the electrode strips in isolation, while the left-hand side depicts the backside of the inner sleeve with the electrode strips mounted on the inner sleeve.

### DETAILED DESCRIPTION

In embodiments of electrode sleeves such as some illustrative disclosed in U.S. Pub. No. 2018/0154133, electrodes are arranged in a fixed grid or as fixed parallel strips arranged to encircle an arm. These arrangements provide little flexibility in designing the electrode configuration for a specific task. For example, the electrodes are not necessarily well aligned with underlying musculature of the arm. Furthermore, the same electronics may be employed to drive FES or NMES and also to read EMG signals. While time division multiplexing and isolation circuitry can permit such dual use of an electrodes sleeve, there is still some possibility of crosstalk in which the stimulation applied during FES or NMES interferes with EMG readout. Yet a further difficulty is that the sleeve must have a number and distribution of electrodes that is sufficient to provide stimulation and/or EMG readout over the entire surface area of the arm. This can be costly in terms of materials, and results in complex circuitry to enable individual addressing of each and every electrode, and may be overly complex for tasks in which only a smaller region of the arm area needs to be stimulated or have EMG readout.

Disclosed herein are improved electrode sleeves for use in EMG, FES, and/or NMES that provide configurable electrode arrangements.

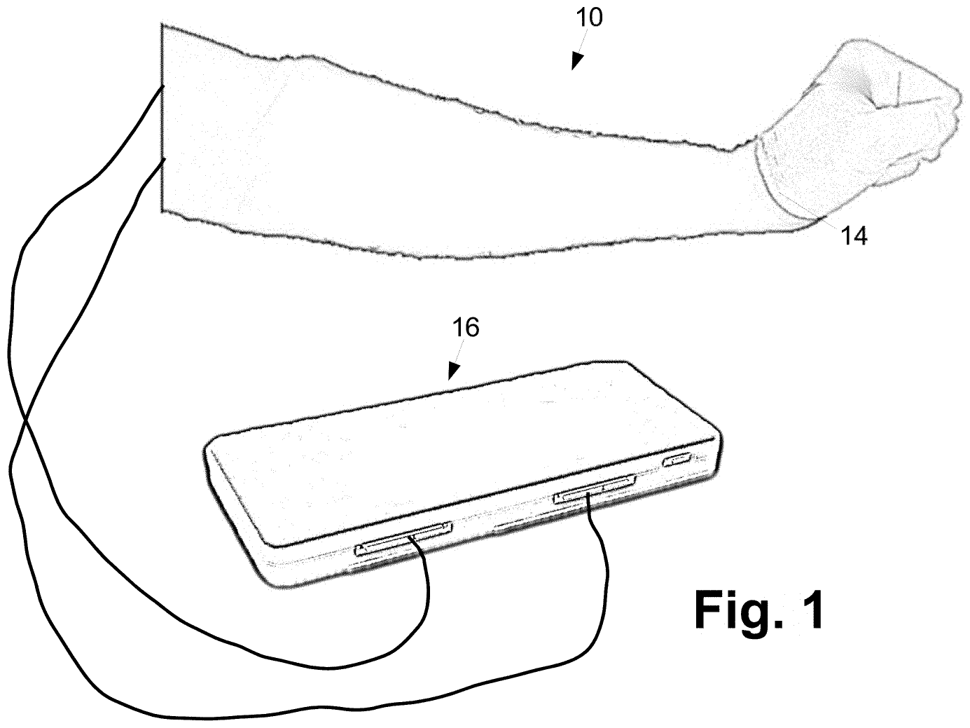

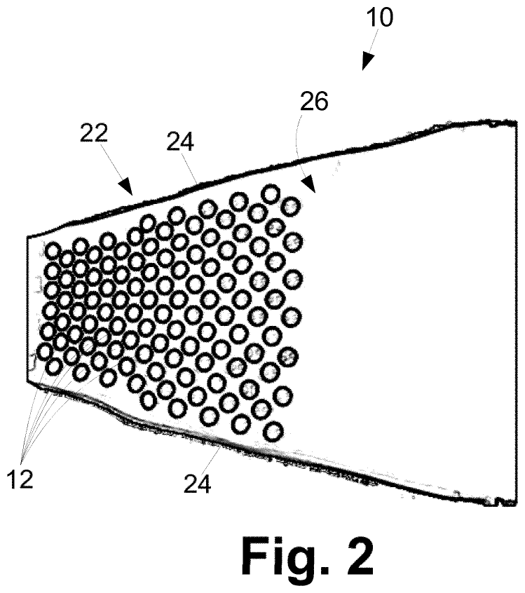

With reference to FIGS. 1 and 2, an illustrative device is shown for use in performing functional electrical stimulation (FES), in performing neuromuscular electrical stimulation (NMES), and/or in receiving electromyography (EMG) signals. The device includes a sleeve 10 and electrodes 12 (see FIG. 2). FIG. 1 shows a perspective view of the device in combination with driving/control hardware. The sleeve is designed to be wrapped around the arm 14 of the user. FIG. 2 shows the unwrapped (i.e., open) sleeve 10 with the electrodes 12 which contact the skin of the arm 14 when the sleeve is wrapped onto the arm 14 as in FIG. 1. An electronics module 16 is provided, which operates the sleeve 10 to perform FES, NMES, and/or readout of EMG. For FES or NMES, the electronics module 16 energizes selected subsets of the electrodes 12 to stimulate FES or NMES. The stimulation can result in muscle contraction leading to induced movement, or can produce somatostimulation so as to simulate a sensation of touch, heat, or the like. For EMG readout, the electronics module 16 reads voltages on the electrodes 12 to measure EMG produced by musculature of the arm 14. It is also noted that some of the electronics may be integrated into the sleeve 10.

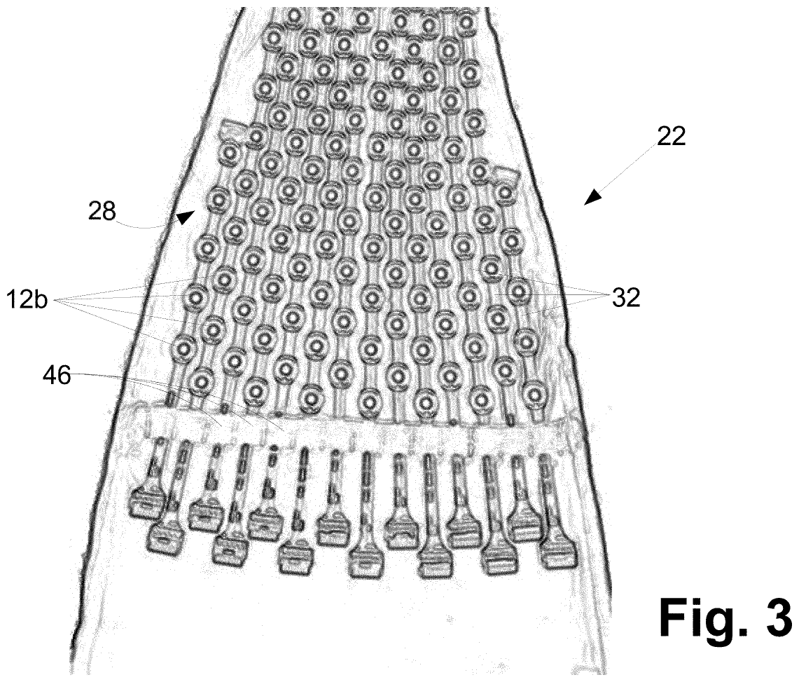

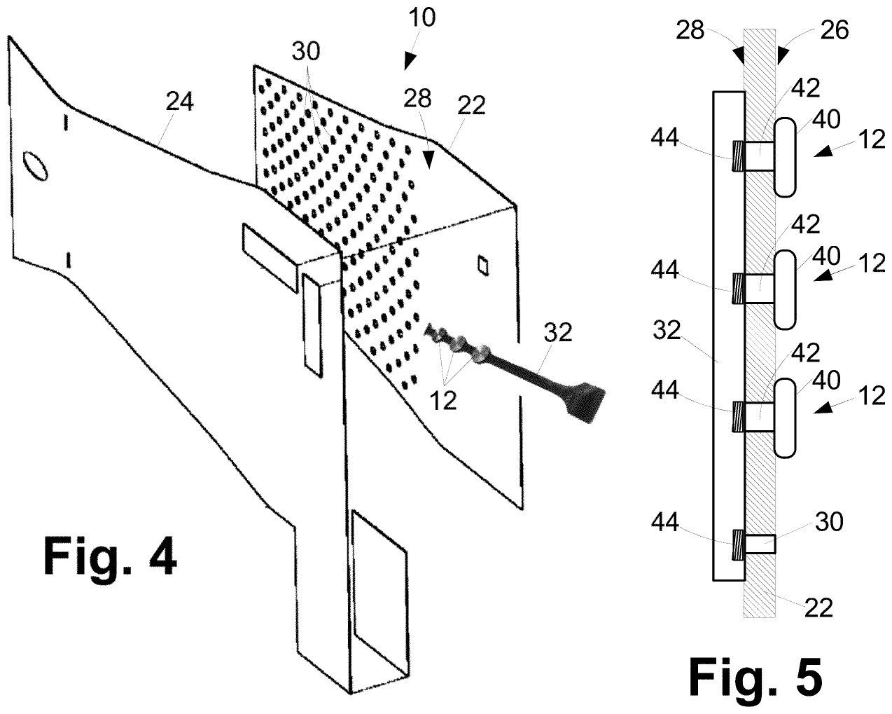

With reference to FIG. 2 and with further reference to FIGS. 3, 4, and 5, an illustrative implementation of the mounting of the electrodes 12 is described. As best seen in diagrammatic FIG. 4, the sleeve 10 in this embodiment includes an inner sleeve 22 that is in contact with the skin of the human arm when the sleeve 10 is worn on the human arm, and an outer sleeve 24 disposed over the inner sleeve 22 when the sleeve is worn on the human arm. Thus, the view of the open sleeve in FIG. 2 more particularly depicts the inner sleeve 22. The inner sleeve 22 has an exposed side 26 positioned to contact skin of the human arm when the sleeve 10 is worn on the human arm and an opposite backside 28 facing the outer sleeve. FIG. 2 depicts the exposed side 26 of the inner sleeve 22, that is, the side of the inner sleeve 22 that contacts the skin. FIG. 3 depicts the backside 28 of the inner sleeve 22, that is, the side 28 of the inner sleeve 22 that faces the outer sleeve 24. To further clarify, the exposed side 26 and the backside 28 are the two opposite principal sides of the inner sleeve 22.

As seen in the exploded view of FIG. 4, the inner sleeve 22 has openings 30. In the assembled device, the electrodes 12 are disposed in the openings 30 of the inner sleeve 22 of the sleeve 10. More particularly, the electrodes 12 are mounted on circuit boards 32 to form electrode assemblies that are connected to the inner sleeve 22. The circuit boards 32 of the electrode assemblies are disposed between the inner sleeve 22 and the outer sleeve 24 as diagrammatically shown in FIG. 4 for a single circuit board 32, and the electrodes 12 are inserted through the openings 30 of the inner sleeve 22 to contact skin of the human arm when the sleeve is worn on the human arm. FIG. 3 depicting the backside 28 of the inner sleeve 22 shows the circuit boards 32 and the backsides 12b of the electrodes (where the electrodes 12 are seen in FIG. 2 which shows the exposed side 26 of the inner sleeve 22). The openings 30 may be reinforced with hole reinforcements, e.g. a vinyl (or more generally electrically insulating) ring concentrically placed around each opening 30.

With reference to FIG. 5, a side-sectional view is shown depicting the attachment of the electrodes 12 to the inner sleeve 22. In this non-limiting illustrative implementation, the electrodes 12 comprise disk portions 40 and connecting portions 42 of narrower diameter than the disk portions 40. The connecting portions 42 are connected with the circuit board 32. Optionally, this is by way of a removable connection, such as a threaded opening 44 in the circuit board—in this approach, the end of the connecting portion 42 that connects with the circuit board 32 has an exterior threading that mates with the inside threading of the threaded opening 44 to allow the electrode 12 to be screwed into the circuit board 32. To illustrate the removability, the bottom threaded opening 44 is shown without an installed electrode. Instead of the threaded openings 44, other mating mechanisms can be employed to detachably connect the connecting portion 42 of the electrode 12 to the circuit board 32, such as a frictional/pressure fit, a snap-lock fit, or so forth.

Each circuit board 32 and the electrodes 12 mounted on the circuit board 32 (by way of connecting portions 42) form an assembly that is referred to herein as an electrode strip. As best seen in FIG. 3, the circuit boards 12 are linear circuit boards 12, that is, have a large aspect ratio forming a strip with a single row of electrodes 12 mounted, that is, a one-dimensional array or linear array of electrodes 12 mounted. The electrode strip is secured to the inner sleeve 22 at least in part by the electrodes 12 passing through the openings 30 of the inner sleeve 22. The inner sleeve 22 preferably has sufficient elasticity to allow the opening 30 to expand to allow the disk portion 40 to pass through. Once through, the connecting portion 42 of the electrode 12 lies inside the opening 30 (which may be slightly expanded if the diameter of the connecting portion 42 is larger than the relaxed diameter of the opening 30), and the inner sleeve 22 is effectively secured between the disk portions 40 and the circuit board 32, as seen in FIG. 5.

Alternatively, if the inner sleeve 22 is not sufficiently elastic for the electrode disk 40 to pass through the opening 30, then the electrode strip can be installed on the inner sleeve 22 by first aligning the threaded opening 44 with the opening 30 of the inner sleeve 22, and then passing the narrower connecting portion 42 from the exposed side 26 to engage and thread into the threaded opening 44. This can be more tedious, however, as each successive threaded opening 44 of the circuit board 32 must be aligned on the backside 28 of the inner sleeve 22 and then the electrode 12 must be installed from the opposite exposed side 26.

Optionally, in addition to the electrodes 12 passing through the openings 30 providing for securing the electrode strips 12, 62 to the inner sleeve 22, the inner sleeve 22 (and more particularly the backside 28 of the inner sleeve 22) further includes optional elastic loops 46 (further) securing the linear circuit boards 32 of the electrode strips to the inner sleeve 22. Preferably, the linear circuit boards 32 have some flexibility to permit deformation to align with the profile of the arm 14 in the worn state (FIG. 1).

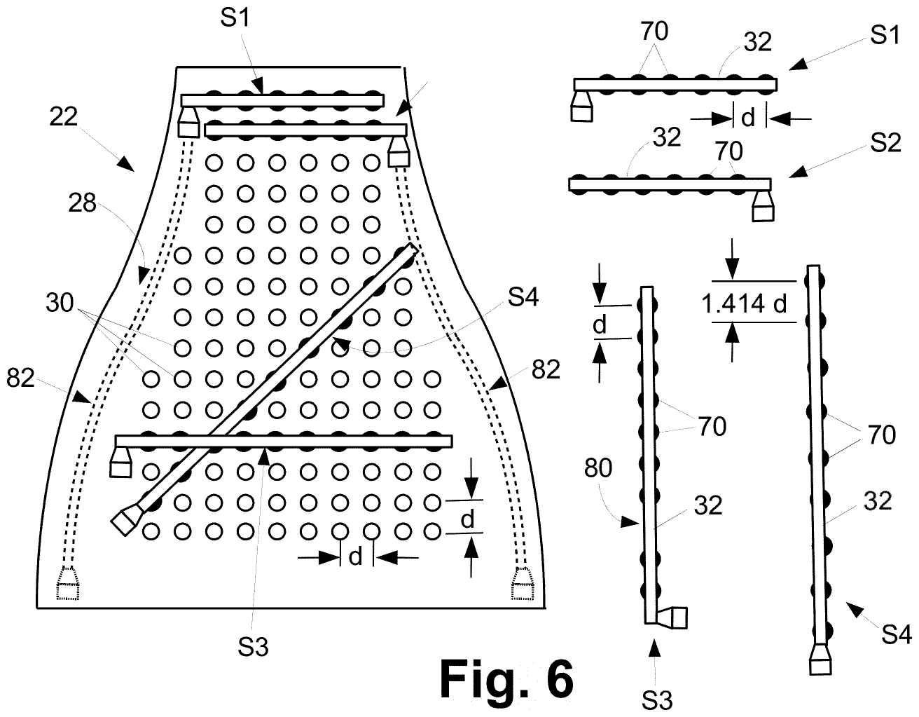

With reference now to FIG. 6, in some embodiments the openings 30 in the inner sleeve 22 form a rectilinear grid with the openings 30 being spaced by a horizontal spacing d and vertical spacing d. The electrode strips then have the electrodes 12 spaced apart by the distance d along the linear circuit board 32. Any given electrode strip can then be mounted on the inner sleeve 22 in either a horizontal orientation or a transverse vertical orientation (or, more generally, in either of the two orthogonal directions defined by the rectilinear grid of the openings 30). In the illustrative example of FIG. 6, there are two strips S1, S2 with six electrodes 70 each with spacing d along the linear circuit board 32. These electrode strips 51, S2 are shown in isolation on the righthand side of FIG. 6, and in the left-hand side of FIG. 6 are shown mounted on a diagrammatic representation of the backside 28 of the inner sleeve 22 near the wrist of the user. This may be a suitable performing NMES or FES on the muscles proximate to the wrist, or for reading EMG from those muscles. If this is the only area to be stimulated or have EMG readout for a given user or user session, then the remaining openings 30 can be left unfilled with electrodes, thereby reducing cost. If more electrodes (or differently positioned) electrodes are needed in a subsequent session then they can be added.

With continuing reference to FIG. 6, for illustrative purposes two additional electrode strips S3 and S4 are shown in isolation (righthand side of FIG. 6) and mounted on the inner sleeve 22 as shown in the left-hand side of FIG. 6. The electrode strip S3 has ten electrodes 70 and is mounted horizontally (or, more generally, along the transverse direction to the direction the electrodes strips S1, S2 are mounted). The electrode strip S4 is mounted diagonally, that is, at a 45-degree angle to the direction of the electrode strip S3. To accomplish this diagonal mounting with the rectilinear grid of openings 30 with spacing d in both directions, the spacing of the electrodes 70 on the electrode strip S4 is \sqrt{2}×d which equals 1.414×d.

More generally, if the openings 30 are arranged in a rectilinear grid that has different spacings d_{H} and d_{V} in the horizontal and vertical directions, respectively, then: (i) the spacing of electrodes on an electrodes strip for mounting vertically is d_{V}; (ii) the spacing of electrodes on an electrodes strip for mounting horizontally is d_{H}; and (iii) the spacing of electrodes on an electrodes strip for mounting diagonally is \sqrt{(d_{H})²+(d_{V})²}. Even more generally, it will be appreciated that the openings 30 could be arranged in some other periodic pattern besides a rectilinear pattern, such as a pattern with six-fold symmetry (i.e., hexagonal) or with eight-fold symmetry (i.e. octagonal), and simple geometric analysis can be done to determine the electrode spacings for electrode strips mounted in various orientations in such non-rectilinear grids.

With continuing reference to FIG. 6, if two electrode strips cross such that they would both have an electrode mounted in the same opening of the pattern, then one electrode strip preferably has that electrode removed. This is illustrated in FIG. 6, in which the horizontal electrode strip S3 is placed over the diagonal electrode strip S4. To accommodate the crossing, the overlaid electrode strip S3 has one electrode removed, leaving an empty electrode slot 80. Such removal can, for example, be implemented by way of a removable connection of the electrodes 70 to the linear circuit boards 32, such by way of the illustrative threaded opening 44 in the circuit board 32 previously described with reference to FIG. 5.

One difficulty with such electrode strips being configurably positioned on the inner sleeve 22 is providing electrical connection to the strips. This could be done using flexible electrical wires (not shown). To reduce the length of such wires, optionally one or more electrical buses 82 (two such buses shown in FIG. 6) are provided at the periphery of the inner sleeve 22.

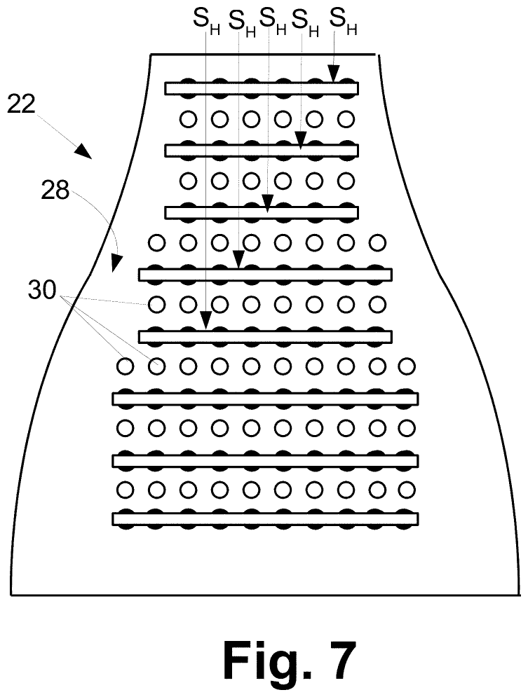

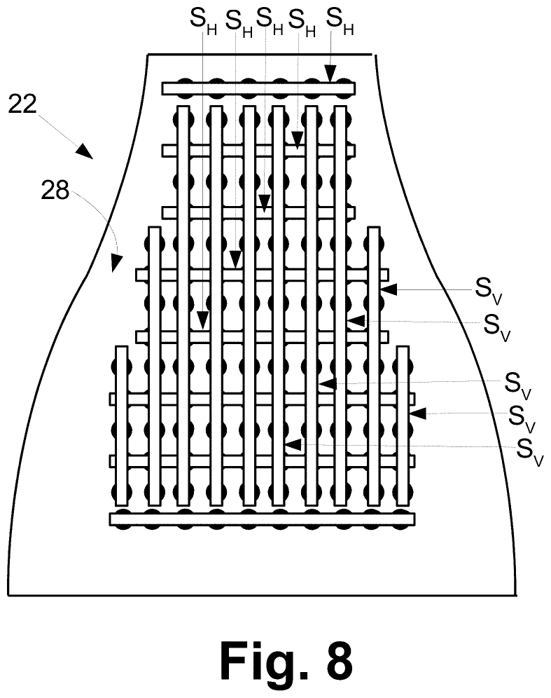

With reference to FIGS. 7 and 8, in another application of the configurably positioned electrode strips, a design is employed in which there is a set of horizontally oriented electrode strips S_{H} mounted in every other horizontal row of openings 30, as shown in FIG. 7. Thereafter, a set of vertically oriented electrode strips S_{V} are mounted in every vertical row of openings 30, as shown in FIG. 8. As can be seen in FIG. 7, the number of electrodes the horizontal strips S_{H} is reduced for horizontal rows closer to the wrist to accommodate the narrowing of the inner sleeve 22 closer to the wrist. Likewise, the number of electrodes the vertical strips S_{V} is reduced for horizontal rows closer to the left and right sides of the inner sleeve 22 to accommodate the shape of the inner sleeve 22. In this example, to accommodate the overlaps between the horizontally oriented electrode strips S_{H} and the vertically oriented electrode strips S_{V}, the overlaid vertically oriented electrode strips S_{V} have electrodes removed or omitted at the crossing locations, as seen in FIG. 8.

It will be appreciated that the opposite approach could be used, e.g. the vertically oriented strips could have all their electrodes but be mounted in every other vertical row of openings 30, and the horizontal strips can be placed over those vertical strips in every horizontal row, but with the overlaid horizontal strips having electrodes removed or omitted at the crossing locations.

With continuing reference to FIGS. 7 and 8 and further reference back to FIG. 3, this approach of having crossing horizontal and vertical electrode strips S_{H} and S_{V} has certain advantages over an embodiment such as that of FIG. 3 in which all linear electrode strips are oriented in the same direction (e.g. vertical in the example of FIG. 3). For example, if both stimulation (i.e. FES or NMES) and EMG readout are to be performed in the same area of the arm, then in the embodiment of FIG. 3 the electronics module 16 (see FIG. 1) must be capable of rapidly switching all of the electrode strips between stimulate and EMG readout modes. This entails operating relays or the like to isolate the stimulator from the electrode strip during the EMG readout phase, and to isolate the sensitive EMG readout electronics during the stimulation phase. This increases complexity of the electronics module 16, and can introduce artifacts into the EMG readout phase from residual power from the stimulation phase.

By comparison, in the embodiment of FIGS. 7 and 8, one set of electrode strips (e.g., the horizontal electrode strips S_{H}) can be used only for EMG readout while the other set of electrode strips (e.g., the vertical electrode strips S_{V}) can be used only for stimulation (or vice versa). The electronics module 16 does not need to switch any of the electrode strips from the stimulation mode to the EMG readout mode or vice versa.

In the embodiments of FIGS. 6-8, the openings 30 are assumed to be arranged in a regular pattern, such as the illustrative four-fold symmetric rectilinear pattern, or a hexagonal pattern, or an octagonal pattern, or so forth. This allows for the same sleeve 10 made up of the inner and outer sleeves 22, 24 (see FIG. 4) to have its electrodes be reconfigured for a specific user or specific user session. However, the inner and outer sleeves 22, 24 may be relatively inexpensive, especially since the electrode strips are removable components.

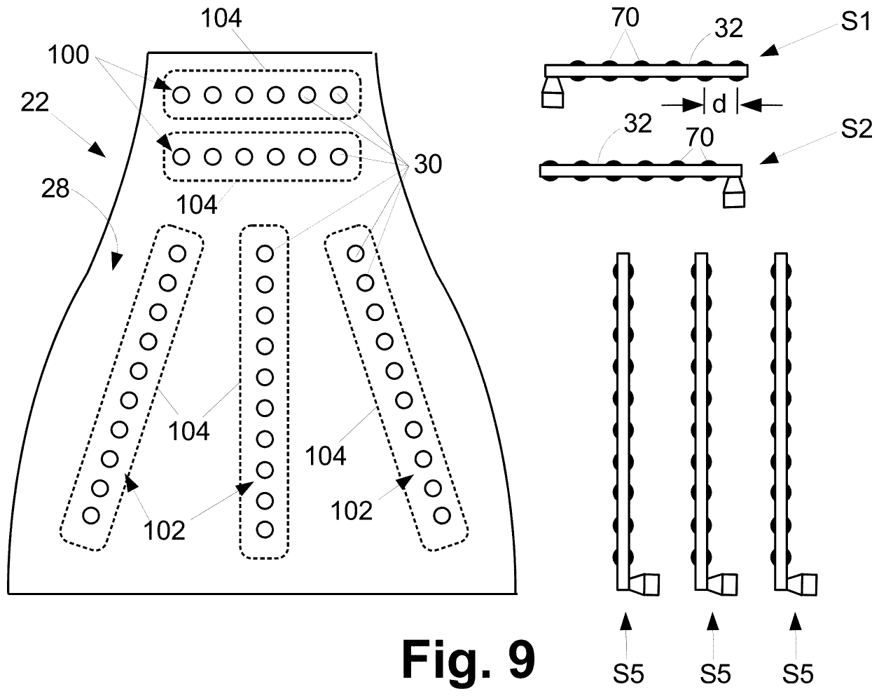

Hence, with reference to FIG. 9, in another approach the inner sleeve 22 is designed with openings 30 arranged in a specific and non-configurable way. The illustrative sleeve has two rows 100 of six openings each for mounting electrode strips S1, S2 for energizing the wrist, and three rows 102 of ten openings each arranged to mount electrode strips S5 lengthwise along the arm. Optionally, dedicated pockets 104 (shown in dashed lines in FIG. 9) are provided on the backside surface 28 of the inner sleeve 22 to assist in holding the various electrode strips in place. It will be appreciated that in this approach, there may be a number of different sleeves with inner sleeves 22 having different patterns of openings 30 for mounting different arrangements of electrode strips, thus providing electrode configurability by choosing the correct sleeve for a given task, although a given sleeve does not have electrode configurability.

The illustrative embodiments are directed to arm sleeves extending over the forearm from (or above) the elbow to (or over) the wrist. More generally, the arm sleeves may additionally or alternatively extend over the upper arm and/or wrist. Even more generally, the device may comprise a wearable garment, such as the illustrative sleeve, a legging that is worn on the leg of the person, a wearable vest or chest band that is worn on the torso and/or abdomen of the person, and/or so forth, with configurable electrodes as disclosed herein. It is contemplated for the garment to cover multiple limbs, e.g. left and right sleeves left and right arms, respectively, which are connected to a common electronics module 48 to provide coordinated FES, NMES, or EMG readout for both left and right arms.

The disclosed sleeve or other wearable garment with configurable electrodes may be employed for various tasks, such as providing somatostimulation to enhance the immersive environment in virtual reality (VR) or augmented reality (AR) systems, to provide somatostimulation and/or force feedback in gaming systems, to provide NMES or FES for providing medical therapy to stroke victims, persons with partial or total paralysis due to a spinal cord injury, and/or so forth, and/or to provide EMG monitoring of musculature affected by such medical conditions, and/or so forth.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. A device for use in performing functional electrical stimulation (FES), in performing neuromuscular electrical stimulation (NMES), in providing somatostimulation, and/or in receiving electromyography (EMG) signals, the device comprising:
a sleeve sized and shaped to be worn on a human arm and comprising an inner sleeve and an outer sleeve wherein the inner sleeve has openings formed therein and has an exposed side positioned to contact skin of the human arm when the sleeve is worn on the human arm and an opposite backside facing the outer sleeve; and
electrode strips each comprising a linear circuit board on which a row of electrodes is mounted, the electrode strips being mounted on the inner sleeve with the linear circuit boards disposed on the backside of the inner sleeve between the inner sleeve and the outer sleeve and the electrodes passing through the openings of the inner sleeve so as to be positioned to contact skin of the human arm when the sleeve is worn on the human arm.

2. The device of claim 1 wherein:
the electrodes comprise disk portions and connecting portions of narrower diameter than the disk portions, and the electrodes pass through the openings of the inner sleeve with the disk portions disposed on the exposed side of the inner sleeve and the connecting portions disposed in the openings of the inner sleeve.

3. The device of claim 1 wherein:
the electrodes comprise disk portions and connecting portions of narrower diameter than the disk portions, the connecting portions being detachably connected with the circuit boards.

4. The device of claim 1 wherein the openings of the inner sleeve are arranged in a periodic pattern and the electrode strips are configured to be mounted to the inner sleeve in different orientations by selectively inserting the electrodes through selected openings of the inner sleeve.

5. The device of claim 1 wherein:
the openings of the inner sleeve are arranged in a rectilinear pattern having a spacing of the openings of d_{H} in a first direction of the rectilinear pattern and a spacing of the openings of d_{V} in a second direction of the rectilinear pattern transverse to the first direction, and
the electrode strips include first electrode strips in which the electrodes of the row of electrodes are spaced apart by the distance d_{H} and are configured to be mounted to the inner sleeve in the first direction, and
the electrode strips include second electrode strips in which the electrodes of the row of electrodes are spaced apart by the distance d_{V} and are configured to be mounted to the inner sleeve in the second direction.

6. The device of claim 5 wherein the electrode strips include diagonal electrode strips in which the electrodes of the row of electrodes are spaced apart by the distance \sqrt{(d_{H})²+(d_{V})²} and are configured to be mounted to the inner sleeve in a diagonal direction.

7. The device of claim 1 wherein:
the openings of the inner sleeve are arranged in a rectilinear pattern having a spacing of the openings of d in both a first direction of the rectilinear pattern and a second direction of the rectilinear pattern transverse to the first direction, and
the electrode strips include electrode strips in which the electrodes of the row of electrodes are spaced apart by the distance d and are configured to be mounted to the inner sleeve in either the first direction or the second direction.

8. The device of claim 7 wherein the electrode strips include diagonal electrode strips in which the electrodes of the row of electrodes are spaced apart by the distance \sqrt{2}×d and are configured to be mounted to the inner sleeve in a diagonal direction.

9. The device of claim 1 wherein the openings of the inner sleeve are arranged in a plurality of rows of openings and the electrode strips are configured to be mounted to the inner sleeve aligned with and mounted to the rows of openings.

10. A method of assembling a device comprising a sleeve having an inner sleeve and an outer sleeve wherein the inner sleeve has an exposed side positioned to contact skin of the human arm when the sleeve is worn on the human arm and an opposite backside facing the outer sleeve, the device being for use in performing functional electrical stimulation (FES), in performing neuromuscular electrical stimulation (NMES), in applying somatostimulation, and/or in receiving electromyography (EMG) signals, the method comprising:
securing electrode strips each comprising a linear circuit board on which a row of electrodes is mounted to the inner sleeve;
wherein the electrode strips are secured to the inner sleeve with the linear circuit boards disposed on the backside of the inner sleeve between the inner sleeve and the outer sleeve and the electrodes passing through the openings of the inner sleeve so as to be positioned to contact skin of the human arm when the sleeve is worn on the human arm.

11. The method of claim 10 wherein:
the electrodes comprise disk portions and connecting portions of narrower diameter than the disk portions, and one of:
(i) the securing of the electrode strips to the inner sleeve comprises passing the disk portions of the electrodes through the openings of the inner sleeve so that the connecting portions are disposed in the openings of the inner sleeve, or
(ii) the connecting portions are detachably connected with the circuit boards and the securing of the electrode strips to the inner sleeve comprises passing the connecting portions through the openings of the inner sleeve and then attaching the connecting portions to the linear circuit board.

12. The method of claim 10 wherein the openings of the inner sleeve are arranged in a periodic pattern and the securing of the electrode strips to the inner sleeve includes securing the electrode strips to the inner sleeve in different orientations aligned with different directions of the periodic pattern.

13. The method of claim 10 wherein:
the openings of the inner sleeve are arranged in a rectilinear pattern having a spacing of the openings of d_{H} in a first direction of the rectilinear pattern and a spacing of the openings of d_{V} in a second direction of the rectilinear pattern transverse to the first direction, and the securing of the electrode strips to the inner sleeve includes:
securing first electrode strips to the inner sleeve in which the electrodes of the rows of electrodes of the first electrode strips are spaced apart by the distance d_{H} and the first electrode strips are secured to the inner sleeve oriented in the first direction, and
securing second electrode strips to the inner sleeve in which the electrodes of the rows of electrodes of the second electrode strips are spaced apart by the distance d_{V} and the second electrode strips are secured to the inner sleeve oriented in the second direction.

14. The method of claim 13 wherein the securing of the electrode strips to the inner sleeve further includes:
securing diagonal electrode strips to the inner sleeve in which the electrodes of the rows of electrodes of the diagonal electrode strips are spaced apart by the distance \sqrt{(d_{H})²+(d_{V})²} and the diagonal electrode strips are secured to the inner sleeve oriented in a diagonal direction.

15. The method of claim 10 wherein:
the openings of the inner sleeve are arranged in a rectilinear pattern having a spacing of the openings of d in both a first direction of the rectilinear pattern and a second direction of the rectilinear pattern transverse to the first direction, and the securing of the electrode strips to the inner sleeve includes:
securing a first one or more electrode strips to the inner sleeve in which the electrodes of the rows of electrodes of the first one or more electrode strips are spaced apart by the distance d and the first one or more electrode strips are secured to the inner sleeve oriented in the first direction, and
securing second one or more electrode strips to the inner sleeve in which the electrodes of the rows of electrodes of the second one or more electrode strips are spaced apart by the distance d and the second one or more electrode strips are secured to the inner sleeve oriented in the second direction.

16. The method of claim 15 wherein the securing of the electrode strips to the inner sleeve further includes:
securing one or more diagonal electrode strips to the inner sleeve in which the electrodes of the rows of electrodes of the one or more diagonal electrode strips are spaced apart by the distance \sqrt{2}×d and the diagonal electrode strips are secured to the inner sleeve oriented in a diagonal direction.

17. The method of claim 10 wherein the openings of the inner sleeve are arranged in a plurality of rows of openings and the securing of the electrode strips to the inner sleeve includes securing electrode strips to the inner sleeve aligned with and mounted to the rows of openings.

18. A device for use in performing functional electrical stimulation (FES), in performing neuromuscular electrical stimulation (NMES), in applying somatostimulation, and/or in receiving electromyography (EMG) signals, the device comprising:
a sleeve sized and shaped to be worn on a human arm and comprising an inner sleeve and an outer sleeve wherein the inner sleeve has openings formed therein and has an exposed side positioned to contact skin of the human arm when the sleeve is worn on the human arm and an opposite backside facing the outer sleeve; and
electrode strips each comprising a linear circuit board on which a row of electrodes is mounted;
wherein the electrode strips are detachably and selectively mountable to the inner sleeve in a plurality of different orientations; and
wherein the electrode strips are mountable on the inner sleeve with the linear circuit boards disposed on the backside of the inner sleeve between the inner sleeve and the outer sleeve and the electrodes passing through the openings of the inner sleeve so as to be positioned to contact skin of the human arm when the sleeve is worn on the human arm.

19. The device of claim 18 wherein the electrode strips are detachably and selectively mountable to the inner sleeve in a first orientation and in a second orientation transverse to the first orientation.

20. The device of claim 19 wherein the electrode strips are further detachably and selectively mountable to the inner sleeve in a diagonal orientation that is diagonal to the first orientation and is diagonal to the second orientation.
